(19) United States
(12) Patent Application Publication
(10) Pub. No.: US 2022/0202352 A1
(43) Pub. Date: Jun. 30, 2022

(54) MALIGNANT TISSUE DETECTION IN BREASTS AND OTHER SYMMETRICAL BODY PARTS COMPARING DIFFERENT NON-IONIZING SIGNALS, INCLUDING MICROWAVES

(71) Applicant: INTRAVISION SA, Geneve (CH)

(72) Inventor: Hossam Eldine Abou Zeid, Conches (CH)

(73) Assignee: INTRAVISION SA, Geneve (CH)

(21) Appl. No.: 17/136,410

(22) Filed: Dec. 29, 2020

(51) Int. Cl. A61B 5/00; A61B 5/0507

(52) U.S. Cl. CPC A61B 5/4312; A61B 5/0507

(57) ABSTRACT

Methods and systems for detecting anomaly in at least one body part among a pair of symmetrical body parts (such as breasts) are described. A device can receive first parameters corresponding to signals scattered from a first body part among a pair of symmetrical body parts, and can receive second parameters corresponding to signals scattered from a second body part among the pair of symmetrical body parts. The device can perform a first comparison between the first parameters and the second parameters. The device can perform a second comparison between the first parameters and first historical data, and a third comparison between the second parameters and second historical data. The device can, based on at least one of the first, second, and third comparisons, determine a likelihood of a presence of an anomaly, such as malignant tissue, in at least one of the first and second body parts.

### FIELD

The present application relates generally to computers, and computer applications, and more particularly to computer-implemented methods and systems to detect a presence of malignant tissue in symmetrical body parts of a body.

### BACKGROUND

Various examinations to detect cancer for body parts of an individual can provide images showing positions, dimensions, and other attributes of malignant tissues. Symmetrical body parts (e.g., breasts of women) can have similar dielectric characteristics if both body parts have normal healthy body part tissues.

### SUMMARY

In some examples, a method for detecting anomaly in at least one body part among a pair of symmetrical body parts of an individual is generally described. The method can include receiving, by a computer device, a plurality of first parameters corresponding to non-ionizing signals scattered from a first body part among the pair of symmetrical body parts. The method can further include receiving, by the computer device, a plurality of second parameters corresponding to non-ionizing signals scattered from a second body part among the pair of symmetrical body parts. The method can further include comparing, by the computer device, the plurality of first parameters with the plurality of second parameters. The method can further include, based on the comparison, determining, by the computer device, a likelihood of a presence of an anomaly in at least one of the first body part and the second body part.

In some examples, a system for detecting anomaly in at least one body part among a pair of symmetrical body parts of an individual are generally described. The system can include a memory configured to store a set of instructions. The system can further include a processor configured to be in communication with the memory. The processor can be configured to execute the set of instructions stored in the memory to receive a plurality of first parameters corresponding to signals scattered from a first body part among a pair of symmetrical body parts of an individual. The processor can be further configured to execute the set of instructions stored in the memory to receive a plurality of second parameters corresponding to signals scattered from a second body part among the pair of symmetrical body parts. The processor can be further configured to execute the set of instructions stored in the memory to compare the plurality of first parameters with the plurality of second parameters. The processor can be further configured to execute the set of instructions stored in the memory to, based on the comparison, determine a likelihood of a presence of an anomaly in at least one of the first body part and the second body part.

In some examples, an apparatus for detecting anomaly in at least one body part among a pair of symmetrical body parts of an individual is generally described. The apparatus can include a first device and a second device. The second device can be configured to be in communication with the first device. The second device can be configured to emit a signal on a pair of body parts. The pair of body parts can include a first body part and a second body part. The first body part and the second body part can be symmetrical body parts of an individual. The second device can be further configured to receive first scattered signals from the first body part. The second device can be further configured to convert the first scattered signals into a plurality of first parameters. The second device can be further configured to receive second scattered signals from the first body part. The second device can be further configured to convert the second scattered signals into a plurality of second parameters. The first device can be configured to receive the plurality of first parameters from the second device. The first device can be further configured to receive the plurality of second parameters from the second device. The first device can be further configured to compare the plurality of first parameters with the plurality of second parameters. The first device can be further configured to, based on the comparison, determine a likelihood of a presence of an anomaly in at least one of the first body part and the second body part.

In some examples, a method for detecting anomaly in at least one body part among a pair of symmetrical body parts of an individual is generally described. The method can include receiving, by a computer device, a plurality of first parameters corresponding to non-ionizing signals scattered from a first body part among the pair of symmetrical body parts. The method can further include receiving, by the computer device, a plurality of second parameters corresponding to non-ionizing signals scattered from a second body part among the pair of symmetrical body parts. The method can further include comparing the plurality of first parameters with first historical data representing historical values of the plurality of first parameters. The method can further include comparing the plurality of second parameters with second historical data representing historical values of the plurality of second parameters. The method can further include, based on the comparison, determining, by the computer device, a likelihood of a presence of an anomaly in at least one of the first body part and the second body part.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1A illustrates an example computer system that can be utilized to examine a body part in one embodiment.

FIG. 1B illustrates the example computer system of FIG. 1A that can be utilized to examine a pair of symmetrical body parts in one embodiment.

FIG. 2 illustrates an example comparison between a pair of symmetrical body parts in one embodiment.

FIG. 3 illustrates an example image showing a presence of an anomaly in a body part in one embodiment.

FIG. 4 illustrates a flow diagram relating to malignant tissue detection in symmetrical body parts using non-ionizing signals, like for example microwaves, in one embodiment.

FIG. 5 illustrates another flow diagram relating to malignant tissue detection in symmetrical body parts using non-ionizing signals, like for example microwaves, in one embodiment.

FIG. 6 illustrates a schematic of an example computer or processing system that may implement malignant tissue detection in symmetrical body parts using non-ionizing signals, like for example microwaves, in one embodiment.

### DETAILED DESCRIPTION

Among existing cancer detection examinations, some of these examinations can involve equipment that can cause discomfort in the individual, or can expose the individual to relatively harmful situations such as exposure to radiation (e.g., X-rays). Thus, in some examples, medical professionals may recommend less frequent examination, such as once every 12 to 36 months, to minimize the individual's exposure. However, in some examples, shorter time period such as 6 months can be enough time for malignant tissue to develop, and such development can be undetected in an individual during the time period between examinations. Further, for detection techniques such as mammography, patients frequently neglect treatment as monitoring the growth of the malignant tissue requires frequent testing and is difficult with existing methods.

In some examples, if one of the symmetrical body parts includes malignant tissue, then the body part having the malignant tissue can exhibit dielectric characteristics different from the dielectric characteristics of the other (normal healthy) of the symmetrical body parts. For instance, for a pair of breasts, if the left breast includes malignant tissue, then the left breast can exhibit dielectric characteristics different from the dielectric characteristics of the right breast. Some imaging systems can leverage the dielectric differences between malignant and healthy tissue, and their different waveforms scattering to generate an image of the body part to highlight a tumor. However, various factors are critical to implement such imaging systems, particularly to achieve high accuracy in the determination of the size and the position of the tumor to be displayed in an image.

Non-ionizing signals, like for example microwaves, can be used as a relatively risk-free noninvasive imaging system for the detection of malignant tissue in the body parts (e.g., breasts) when compared to mammography. The methods and systems described herein can run a relatively simple and quick test using, for example, a microwave-based system to detect a presence or an absence of malignant tissue in one of the two symmetrical body parts of an individual, such as the breasts of a woman. Microwave waveforms can be sent to the breasts (or other symmetrical body parts), and the microwave waveforms can scatter in a different way if malignant tissue is present. Different measurements between the symmetrical body parts can be analyzed and compared to determine a likelihood of whether malignant tissue is present or not. The result of the analysis can provide a likelihood assessment indicating the likelihood of malignant tissue being present. As one example of the likelihood assessment, the result of the analysis can provide a binary assessment, such as malignant tissue suspected of being present or absent, without using image processing techniques to generate images. Thus, the examination procedure can be simplified, less invasive, and relative quick such that the individual may not need to be exposed to harmful radiation for a prolonged period of time. If the results of the analysis and comparison indicate a relatively high likelihood of a presence of malignant tissue, recommendation can be made to the individual to seek a more detailed diagnosis, such as running a full traditional examination (e.g., mammography) to confirm a presence of a tumor and to determine its position and size.

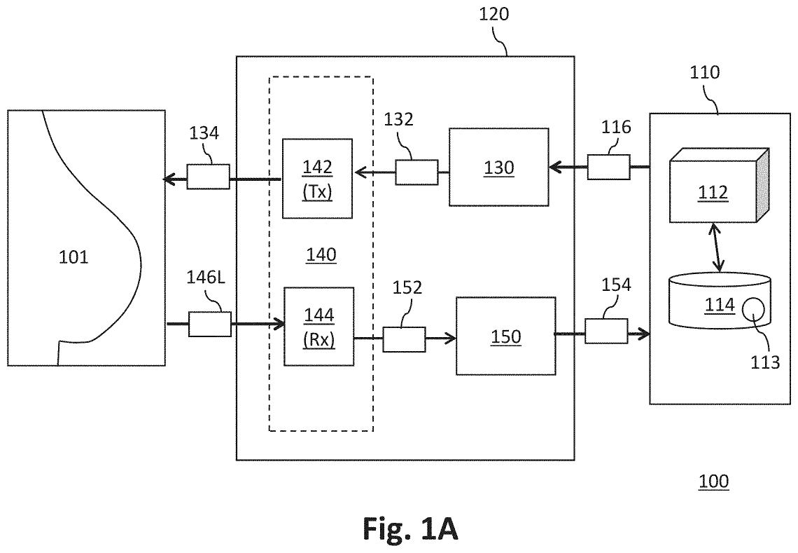

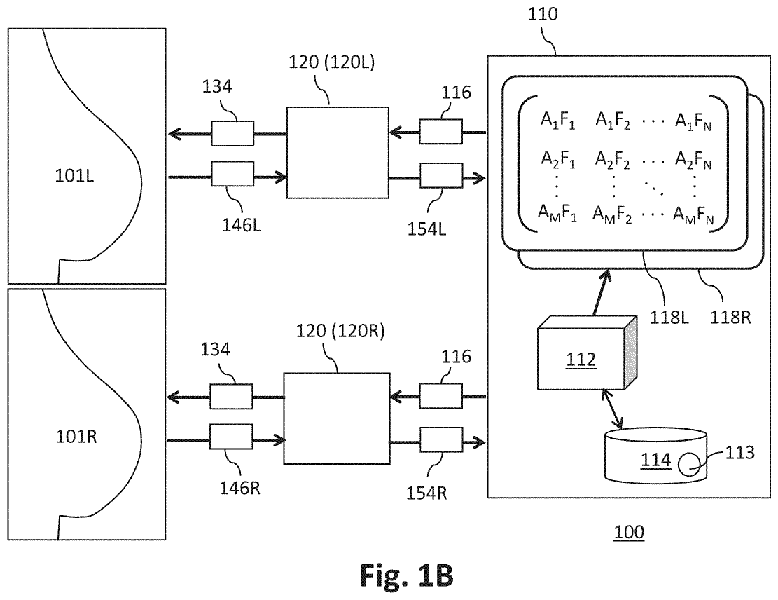

FIG. 1A illustrates an example computer system 100 that can be utilized to examine a body part in one embodiment. The system 100 can include a device 110 and a device 120. The device 110 can be configured to be in communication with the device 120. The device 110 can be a computer device such as a desktop computer, a laptop computer, a tablet device, a server, a mobile phone, a tablet, a wearable device, and/or other types of computer devices. In the example shown in FIG. 1A, the device 110 can include a processor 112 and a memory 114. The device 120 can be, for example, a microwave device configured to generate and emit microwaves on one or more objects, and receive or obtain signals scattered from the one or more objects.

The processor 112 can be configured to control operations of the device 120. For example, the processor 112 can generate a control signal 116 and use the control signal 116 to control the device 120. The control signal can be, for example, an activation signal to activate one or more operations of the device 120, a command indicating instructions to operate the device 120 and a duration of operation or activation, and/or other types of controls and commands. The memory 114 can be configured to store a set of instructions 113. The set of instructions 113 can be, for example, executable code that can be executed by the processor 112 to run an application that can be used by a user to implement the system 100.

In the example shown in FIG. 1A, the device 120 can include a signal generator 130, a signal analyzer 150, a transmitter 142, and a receiver 144. In some examples, the transmitter 142 and the receiver 144 can be components of a transceiver 140. The signal generator 130 can be configured to generate electrical signals 132 and provide the electrical signals 132 to the transmitter 142. The transmitter 142 can include one or more antennas configured to generate radio waves 134 representing the electrical signals 132, and emit the radio waves 134 towards one or more body parts of an individual. The radio waves 134 can be non-ionizing signals, such as, for example, microwaves, that are electromagnetic waves with wavelengths and frequencies in the microwave range. In the example shown in FIG. 1A, the radio waves 134 can be emitted towards a body part 101 of an individual, where the body part can be, for example, a breast of a woman. The body part 101 can be one body part among a pair of symmetrical body parts that can be symmetrical about an axis or plane of the individual, such as being symmetrical about a longitudinal axis of the individual. In an example, the longitudinal axis can be referred to as an axis that runs lengthwise through the human body.

Backscatter (e.g., reflection of waves) can occur in response to the radio waves 134 being emitted on the body part 101. The receiver 144 can include a plurality of antennas configured to receive or obtain the scattered waves including backscattered waves 146 from the body part 101. The receiver 144 can convert the backscattered waves 146 into electrical signals 152. The receiver 144 can send the electrical signals 152 to the signal analyzer 150. The signal analyzer 150 can convert the electrical signals 152 into a plurality of scattering parameters (S-parameters) 154. In an example, the signal analyzer 150 can be configured to measure parameters, such as amplitude, phase, frequency, gain, and/or other parameters of the electrical signals 152, where the measured parameters can be part of the S-parameters 154.

The signal analyzer 150 can send the S-parameters 154 to the device 110. The processor 112 can store the received S-parameters 154 into the memory 114. The system 100 can further emit the radio waves 134 on another body part that is a symmetrical body part to the body part 101, and collect S-parameters from the symmetrical body part. The processor 112 can generate a S-parameter matrix using the S-parameters 154 of the body part 101, and generate another S-parameter matrix with the S-parameters of the symmetrical body part. The processor 112 can compare the two generated S-parameter matrices, and based on the comparison, the processor 112 can determine a likelihood of a presence of an anomaly malignant tissue in one of the body part 101 or the symmetrical body part.

In an example, to perform examination on a pair of symmetrical body parts using the system 100, the device 120 can be set up to examine the two body parts with similar set-up and at the same time, to have the same test conditions for either the patient (e.g., same hormonal status) and the same environment (e.g., temperature, humidity, etc.). For example, the system 100 can be used to examine the left and right breasts of a woman in one visit, with similar set-up and at the same time. In an example embodiment shown in FIG. 1B, the device 120 can emit the radio waves 134 towards a pair of body parts 101L and 101R, where the body parts 101L and 101R can be a left breast and a right breast of a woman, respectively. In some examples, the device 120 can perform the examination on the body parts 101L and 101R on separate instances (e.g., one after another). In some examples, the device 110 can control two pieces or instances of the device 120 (e.g., 120L and 120R) to perform the examination on the body parts 101L and 101R. The devices 120L and 120R can be assigned to examine the body parts 101L and 101R, respectively. The pair of body parts 101L and 101R can be symmetrical about an axis or plane of the individual, such as being symmetrical about a longitudinal axis of the individual. In an example, the longitudinal axis can be referred to as an axis that runs lengthwise through the human body.

Backscatter (e.g., reflection of waves) can occur in response to the radio waves 134 being emitted on the body parts 101L and 101R. The receiver of the device 120L can receive or obtain the scattered waves 146L from the body part 101L. The device 120L can convert the scattered waves 146L into electrical signals, and can convert the electrical signals into a plurality of scattering parameters (S-parameters) 154L. The device 120L can send the S-parameters 154L to the device 110. The processor 112 can store the received S-parameters 154L into the memory 114.

The receiver of the device 120R can receive or obtain the scattered waves 146R from the body part 101R. The device 120R can convert the scattered waves 146R into electrical signals, and can convert the electrical signals into a plurality of scattering parameters (S-parameters) 154R. The device 120R can send the S-parameters 154R to the device 110. The processor 112 can store the received S-parameters 154R into the memory 114.

The processor 112 can generate a S-parameter matrix 118L for the body part 101L using the received S-parameters 154L. The processor 112 can generate a S-parameter matrix 118R for the body part 101R using the received S-parameters 154R. Each one of the S-parameter matrices 118L and 118R can include, for example, values indicating differences between the radio waves 134 and scattered waves 146L, and between the radio waves 134 and 146R, respectively. The values in the S-parameter matrices 118L and 118R that can indicate differences such as differences in amplitude, differences in phase, and/or differences between other parameters. The processor 112 can store the S-parameter matrices 118L and 118R in the memory 114.

Each value among the S-parameter matrices 118L and 118R can correspond to a pair of an antenna identifier (ID) and a frequency value. For example, the receiver (e.g., receiver 144) of the device 120 (or devices 120L and 120R) can include a plurality of antennas, such as M antennas, identified as A₁, A₂, . . . , A_{M}. Each antenna among the M antennas can receive or obtain the scattered wave 146L, where the received scattered wave 146L can have different frequency components, such as N frequencies, ranging from F₁, F₂, . . . , F_{N}. Thus, a value A₁F₁ in the S-parameter matrix 118L can be a value representing a difference between a parameter of the radio wave 134 at the frequency F₁ and the same parameter of the scattered wave 146L received by the antenna A₁ at the frequency F₁.

The processor 112 can be configured to process corresponding entries of the S-parameter matrices 118L and 118R in accordance with the instructions 113. For example, the instructions 113 can include instructions executable by the processor 112 to perform various analysis techniques such as, for example, matrix operations and arithmetic, signal processing (including signal comparison), image processing, regression analysis, classification model analysis, and/or other types of techniques that can be used to analyze the S-parameter matrices 118L and 118R. In an example, the instruction 113 can include instructions executable by the processor 112 to compare corresponding entries of the S-parameter matrices 118L and 118R. The entry A₁F₁ in the S-parameter matrix 118L can be compared with the entry A₁F₁ in the S-parameter 118R, and the entry A₂F₂ in the S-parameter matrix 118L can be compared with the entry A₂F₂ in the S-parameter 118R. In some examples, for each pair of compared entries, the processor 112 can analyze a difference between the compared entries and determine whether the difference indicates an irregularity. For example, the set of instructions 113 can define a difference threshold as a given percentage, such that if the compared entries have a difference of more than the given percentage, then the processor 112 can determine that there is an irregularity between the compared entries. The difference threshold defined in the set of instructions 113 can be arbitrary, and can be adjusted according to a desired implementation of the system 100. For example, if a physician would like to increase a sensitivity of the system 100, the physician can decrease the value of the defined difference threshold. Further, if K pairs of entries are compared, the processor 112 can determine a likelihood of a presence of an anomaly in one or more of the body parts 101L and 101R based on a number of compared entries having irregularities. For example, the set of instructions 113 can define an anomaly threshold, such that if K entries are compared and the compared entries having irregularity is more than the anomaly threshold, the processor 112 can determine that there may be a higher likelihood of an anomaly in one or more of the body parts 101L and 101R. The anomaly threshold can be arbitrary, and can be adjusted according to a desired implementation of the system 100. The comparison of the S-parameter matrices 118L and 118R can allow the system 100 to determine a likelihood of the presence of anomalies and in one specific example, exclude or confirm the presence of anomalies, such as suspected presence of malignant tissue, in one or more of the body parts 101L and 101R.

The memory 114 can store a plurality of historical S-parameter matrices for the body parts 101L and 101R. In an example, the historical S-parameter matrices for the body parts 101L and 101R can include a plurality of historical S-parameter matrices. For example, historical S-parameter matrices for the body part 101L can include a set of historical matrices generated based on signals scattered from the body part 101L during X different examinations in recent Y time period (e.g., 6 months, 1 year, 2 years, etc.). Similarly, the historical S-parameter matrices for the body part 101R can include a set of historical matrices generated based on signals scattered from the body part 101R during the X different examinations in the recent Y time period (e.g., 6 months, 1 year, 2 years, etc.). The processor 112 can further compare the S-parameter matrix 118L with historical S-parameter matrices of the body part 101L, and can compare the S-parameter matrix 118R with historical S-parameter matrices of the body part 101R. The comparison of the S-parameter matrices 118L and 118R with their historical S-parameter matrices can provide further confirmation of a likelihood of a presence or absence of malignant tissue. For example, if the S-parameter matrices 118L and 118R are significantly different, but similar to their historical S-parameter matrices, then the processor 112 can determine that there may be a decreased likelihood of malignant tissue present in the body parts 101L and 101R since there does not appear to be significant changes over time.

In another example, if the S-parameter matrices 118L and 118R are similar, but the S-parameter matrix 118L is different from its historical S-parameter matrices by, for example, approximately D %, and the S-parameter matrix 118R is different from its historical S-parameter matrices by, for example, approximately G %, where G can be significantly different from D, then the processor 112 can determine that there may be an increased likelihood of malignant tissue in the body parts 101R since the S-parameter matrices 118R appears to be changing at a different rate when compared to the S-parameter matrices 118D. In another example, if the S-parameter matrices 118L and 118R are similar, but different from their respective historical S-parameter matrices by the same rate, such as both being different by D %, then the processor 112 can determine that there may be a decreased likelihood of malignant tissue in both the body parts 101L and 101R since both S-parameter matrices appear to be changing over time at the same rate. Note that there may be differences between current S-parameter matrices and previous S-parameter matrices due to factors such as hormone levels, temperature, examination environment, etc. The comparison of the S-parameter matrices (at current time, the current S-parameters) with the historical S-parameter matrices can include comparison with most recent S-parameter matrices (e.g., from the individual's last examination) or with multiple previous S-parameter matrices (e.g., from the previous three visits, five visits, from all visits in the past year, etc.).

In another example embodiment, upon obtaining the S-parameter matrices 118L and 118R, the processor 112 can compare the S-parameter matrix 118L with historical S-parameter matrices of the body part 101L, and can compare the S-parameter matrix 118R with historical S-parameter matrices of the body part 101R. The comparison of the S-parameter matrices 118L and 118R with their historical S-parameter matrices can provide a chronological set of S-parameter data and/or matrices that can reflect, for example, tissue changes of the body parts 101 and 101R over a period of time. In this embodiment, the system 100 can perform analysis on the chronological set of S-parameter data and/or matrices to determine a likelihood of a presence of any anomaly (e.g., suspected malignant tissue), or whether anomaly is present or absent, in one or more of the body parts 101L and 101R without comparing the current S-parameter matrices 118L and 118R with each other.

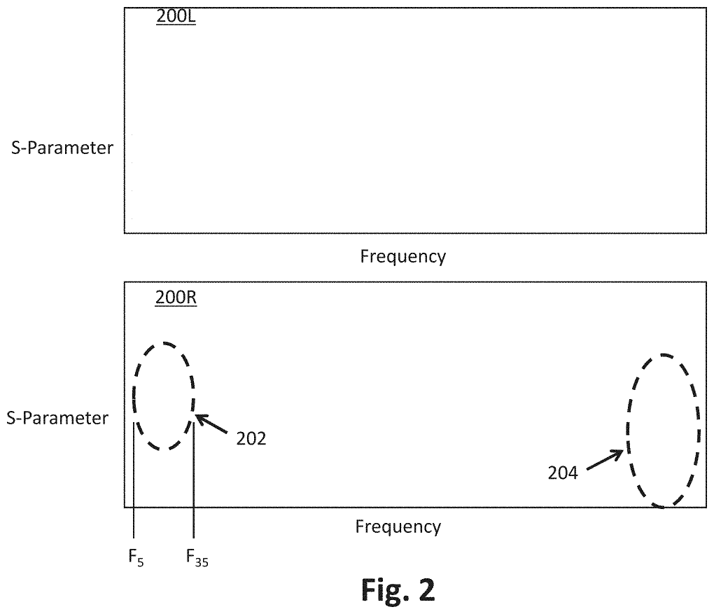

FIG. 2 illustrates an example comparison between a pair of symmetrical body parts (e.g. breasts) in one embodiment. In some examples, the processor 112 can render the S-parameter matrices 118L and 118R into visual objects, such as graphs, and can output the graphs on a user interface of the device 110 for analysis by a user such as a physician or technician. In an example shown in FIG. 2, the processor 112 can render the S-parameter matrices 118L and 118R into graphs 200L and 200R, respectively. In some examples, the processor 112 can compare the S-parameter matrices 118L and 118R by comparing the graphs 200L and 200R. In the example shown in FIG. 2, a relatively notable difference can be identified at frequencies 202 (e.g., from F₅ to F₃₅) and at frequencies 204. Thus, the processor 112 can determine that there is a greater likelihood that at least one of the body parts 101L and 101R has an anomaly, such as malignant tissue.

In an example, symmetrical body parts (e.g., breasts) are similar such that they have similar tissue composition and dielectric characteristics, and can be both affected in the same way by the hormonal status of the patient during the examination using the system 100. Therefore, if no anomaly, such as malignant tissue, is present in either body part, the S-parameter matrices 118L and 118R shall be similar, within limited tolerances. Such results can indicate that the symmetrical body parts are potentially healthy. However, if at least one of the symmetrical body parts have probable malignant tissue, the tissue composition and dielectric characteristics may change in the body part with the anomaly, and the S-parameter matrices can show differences that could be recognizable even at the early stage of the malignant tissue growth. Based on the difference between the S-parameter matrices, a physician or technician can recommend the individual having the potential anomaly to undergo a traditional full check (e.g., mammography, biopsy) to determine the characteristics of the suspected malignant tissue.

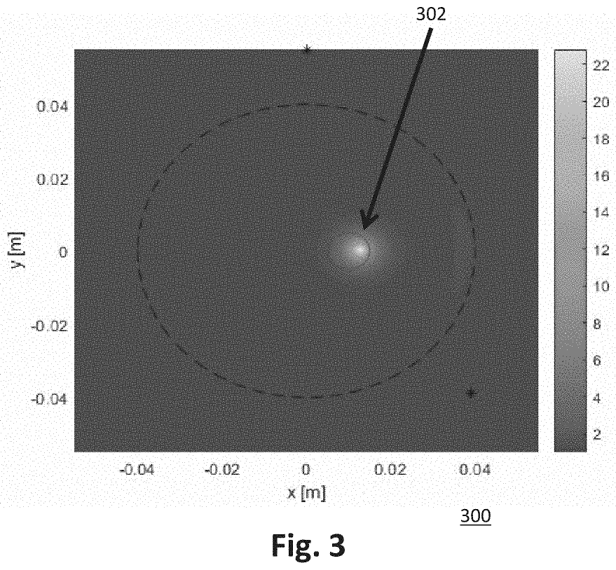

FIG. 3 illustrates an example image showing a presence of an anomaly in a body part (e.g. breast) in one embodiment. In some examples, the set of instructions 113 can include signal processing and/or image processing instructions that can be executable by the processor 112 to generate images based on the S-parameters matrices 118L and 118R. For example, the set of instructions 113 can include executable code relating to multiple signal classification (MUSIC) algorithm, and the processor can apply the MUSIC algorithm on the S-parameter matrices 118L or 118R to generate one or more images. In an example, if a body part includes anomaly of a suspected malignant tissue, an image, such as the image 300 shown in FIG. 3, can include a spot 302 showing a presence of an anomaly.

In some examples, if the set of instructions 113 uses imaging techniques such as the MUSIC algorithm for detection of malignant tissue in body parts, several issues may need to be addressed. For example, in order to detect relatively small tumors, such as at the early stage of the tumor's development, higher resolution and higher frequencies (e.g., more than 10 GHz) of radio waves may be required. The higher the frequency, the lower the depth of penetration of the waveform into the body part (e.g., breast). Therefore, it might be necessary to increase the power of the transmitted signal or radio waves. However, this increased power can be normally limited by severe regulatory recommendations for the overall radiation power to human body. Another issue to be addressed is that proper coupling dielectric materials between the antennas and the body part's skin may be required, to avoid or to limit signal reflections from the body part's skin that can affect the signal to noise ratio (SNR) of the scattered waves returning to the antennas. Finally it may be difficult to provide a single good-for-all physical structure (e.g., device 120) to accommodates different arrangement of antennas that can be based on different shapes and sizes of the body parts (e.g., breasts) between different individuals.

The methods and systems described in the present disclosure attempts to mitigate these issues by means of methodologies which are able to broadly exclude, or to confirm, the likelihood of a presence of malignant tissue in one of the two breasts (or other symmetrical body parts) of an individual, by analyzing and comparing different measurements of that breast (or body part) and identifying differences and changes among them, without generating images of that breast (or body part), such as the ones obtained using the MUSIC algorithm or other imaging techniques.

The methods and systems described in the present disclosure may be used as a supplemental examination that can provide reasonable reliability, small radiation power and the noninvasive set up, such that it can be frequently repeated without affecting the health of the patient, be relatively less invasive, safer, and can be conducted more frequently. The methods and systems described in the present disclosure may complement existing examinations such as mammography or other existing diagnostic tools, such as echography or tomography, such that breast (or body part) examination can be frequently and safely repeated. In principle, if the result of examination is negative, the individual may not need any additional check, whilst, if the result is positive, the individual may be asked to run a traditional full check, to get a detailed description of the suspected malignant tissue.

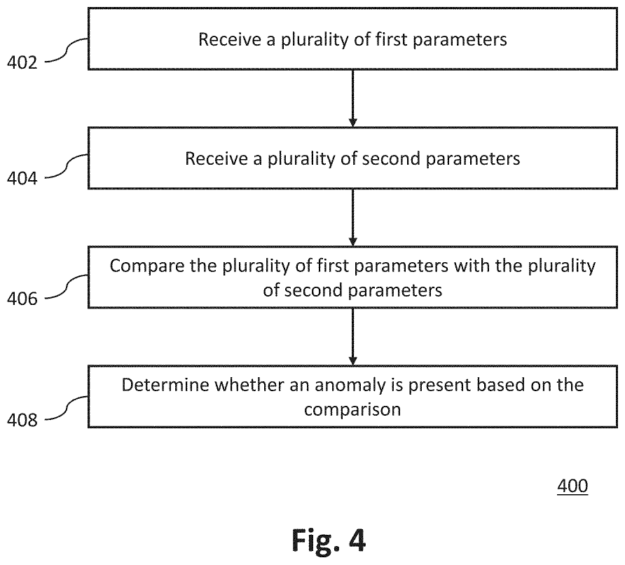

FIG. 4 illustrates a flow diagram relating to malignant tissue detection in symmetrical body parts using non-ionizing signals, like for example microwaves, in one embodiment. The process 400 in FIG. 4 may be implemented using, for example, computer system 100 discussed above. An example process may include one or more operations, actions, or functions as illustrated by one or more of blocks 402, 404, 406, and/or 408. Although illustrated as discrete blocks, various blocks may be divided into additional blocks, combined into fewer blocks, eliminated, or performed in parallel, depending on the desired implementation.

The process 400 can begin at block 402, where a computer device can receive or retrieve a plurality of first parameters corresponding to signals scattered from a first body part among a pair of symmetrical body parts of an individual. The process 400 can proceed from block 402 to block 404, where the computer device can receive or retrieve a plurality of second parameters corresponding to signals scattered from a second body part among the pair of symmetrical body parts. In some examples, the pair of symmetrical body parts can be a pair of breasts of a woman.

The process 400 can proceed from block 404 to block 406, where the computer device can compare the plurality of first parameters with the plurality of second parameters. In some examples, the comparison can include generating a first matrix representing the plurality of first parameters, generating a second matrix representing the plurality of second parameters, and comparing corresponding entries of the first matrix and the second matrix. The process 400 can proceed from block 406 to block 408, where the computer device can, based on the comparison, determine a likelihood of a presence of an anomaly in at least one of the first body part and the second body part. In some examples, the anomaly can be the presence of malignant tissue.

In some examples, in response to a difference between the plurality of first parameters and the plurality of second parameters being less than a threshold, the computer device can determine that there is a higher likelihood of the anomaly being absent from the pair of symmetrical body parts. In response to a difference between the plurality of first parameters and the plurality of second parameters being greater than a threshold, the computer device can determine that there is a higher likelihood of the anomaly being present in at least one body part of the pair of symmetrical body parts.

In some examples, the computer device can further compare the plurality of first parameters with first historical data representing historical values of the plurality of first parameters. The computer device can further compare the plurality of second parameters with second historical data representing historical values of the plurality of second parameters. The determination of the likelihood of the presence of the anomaly can be further based on the comparison of the plurality of first parameters with the first historical data and the comparison of the plurality of second parameters with the second historical data.

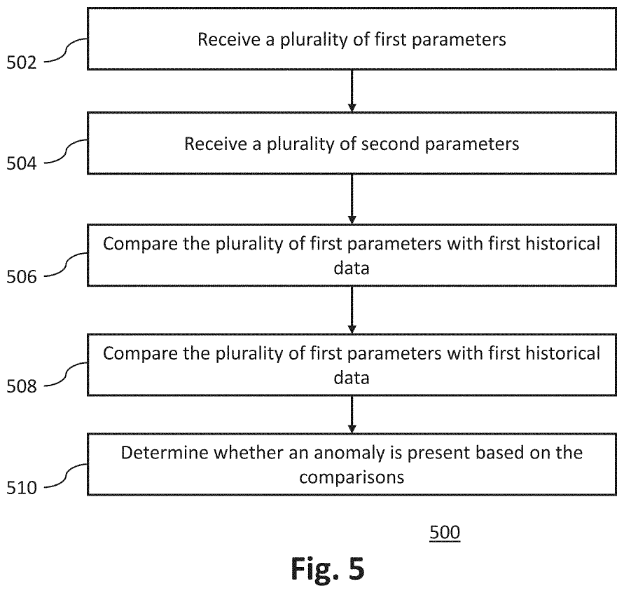

FIG. 5 illustrates another flow diagram relating to malignant tissue detection in symmetrical body parts using non-ionizing signals, like for example microwaves, in one embodiment. The process 500 in FIG. 5 may be implemented using, for example, computer system 100 discussed above. An example process may include one or more operations, actions, or functions as illustrated by one or more of blocks 502, 504, 506, 508, and/or 510. Although illustrated as discrete blocks, various blocks may be divided into additional blocks, combined into fewer blocks, eliminated, or performed in parallel, depending on the desired implementation.

The process 500 can begin at block 502, where a computer device can receive or retrieve a plurality of first parameters corresponding to signals scattered from a first body part among a pair of symmetrical body parts of an individual. The process 500 can proceed from block 502 to block 504, where the computer device can receive or retrieve a plurality of second parameters corresponding to signals scattered from a second body part among the pair of symmetrical body parts. In some examples, the pair of symmetrical body parts can be a pair of breasts of a woman.

The process 500 can proceed from block 504 to block 506, where the computer device can compare the plurality of first parameters with first historical data representing historical values of the plurality of first parameters. In some examples, the comparison of the plurality of first parameters with first historical data can include generating a first matrix representing the plurality of first parameters, and comparing corresponding entries of the first matrix and the first set of historical matrices.

The process 500 can proceed from block 506 to block 508, where the computer device can compare the plurality of second parameters with second historical data representing historical values of the plurality of second parameters. In some examples, the comparison of the plurality of second parameters with second historical data can include generating a second matrix representing the plurality of second parameters, and comparing corresponding entries of the second matrix and the second set of historical matrices.

The process 500 can proceed from block 508 to block 510, where the computer device can, based on the comparisons, determine a likelihood of a presence of an anomaly in at least one of the first body part and the second body part. In some examples, the anomaly can be a presence of malignant tissue.

In some examples, in response to a difference between the plurality of first parameters and the first historical data being less than a threshold, the computer device can determine that there is a higher likelihood of the anomaly being absent from the first body part. In some examples, in response to a difference between the plurality of second parameters and the second historical data being less than the threshold, the computer device can determine that there is a higher likelihood of the anomaly being absent from the second body part. In some examples, in response to a difference between the plurality of first parameters and the first historical data being greater than the threshold, the computer device can determine that there is a higher likelihood of the anomaly being present in the first body part. In some examples, in response to a difference between the plurality of second parameters and the second historical data being greater than the threshold, the computer device can determine that there is a higher likelihood of the anomaly being present in the second body part.

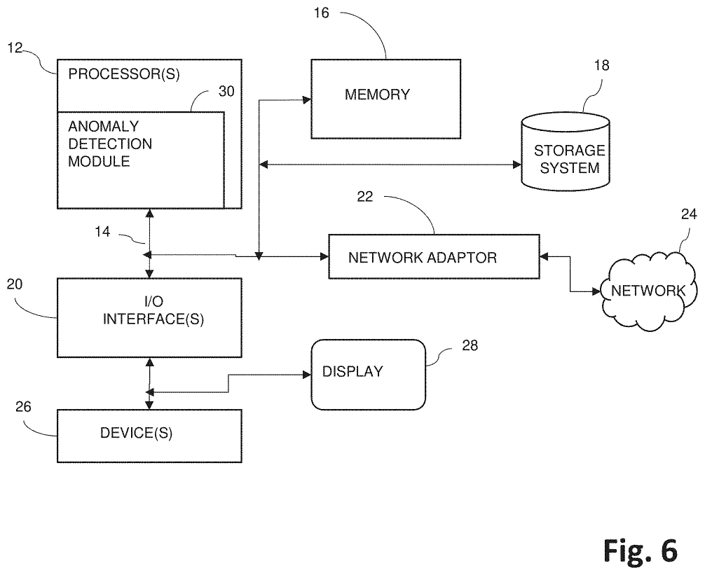

FIG. 6 illustrates a schematic of an example computer or processing system that may implement malignant tissue detection in symmetrical body parts using non-ionizing signals, like for example microwaves, in one embodiment. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of computing systems, environments, and/or configurations that may be suitable for use with the processing system shown in FIG. 6 may include, but are not limited to, personal computer systems, server computer systems, thin clients, thick clients, handheld or laptop devices, mobile phones, tablet computers, wearable devices, virtual reality devices, multiprocessor systems, microprocessor-based systems, set top boxes, programmable consumer electronics, network PCs, minicomputer systems, mainframe computer systems, supercomputers, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units 12, a system memory 16, and a bus 14 that couples various system components including system memory 16 to processor 12. The processor 12 may include a module 30 (e.g., anomaly detection module 30) that performs the methods described herein. The module 30 may be programmed into the integrated circuits of the processor 12, or loaded from system memory 16, storage device 18, or network 24 or combinations thereof.

Bus 14 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus, and universal serial bus (USB).

Computer system may include a variety of computer system readable media. Such media may be any available media that is accessible by computer system, and it may include both volatile and non-volatile media, removable and non-removable media.

System memory 16 can include computer system readable media in the form of volatile memory, such as random access memory (RAM) and/or cache memory or others. Computer system may further include other removable/non-removable, volatile/non-volatile computer system storage media. By way of example only, storage device 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. Other examples of non-volatile memory or storage media can include, for example, flash memory, solid state drive (SSD), magnetoresistive random-access memory (MRAM), In such instances, each can be connected to bus 14 by one or more data media interfaces.

Computer system may also communicate with one or more external devices 26 such as a keyboard, a pointing device, a display 28, etc.; one or more devices that enable a user to interact with computer system; and/or any devices (e.g., network card, modem, etc.) that enable computer system to communicate with one or more other computing devices. Such communication can occur via Input/Output (I/O) interfaces 20.

Still yet, computer system can communicate with one or more networks 24 such as a local area network (LAN), a general wide area network (WAN), WI-FI, Bluetooth, a cellular network (e.g., 3G, 4G, 5G, Long-Term Evolution (LTE)), and/or a public network (e.g., the Internet) via network adapter 22. As depicted, network adapter 22 communicates with the other components of computer system via bus 14. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method for detecting anomaly in at least one body part among a pair of symmetrical body parts of an individual, the method comprising:
receiving, by a computer device, a plurality of first parameters corresponding to non-ionizing signals scattered from a first body part among the pair of symmetrical body parts;
receiving, by the computer device, a plurality of second parameters corresponding to non-ionizing signals scattered from a second body part among the pair of symmetrical body parts;
comparing, by the computer device, the plurality of first parameters with the plurality of second parameters; and
based on the comparison, determining, by the computer device, a likelihood of a presence of an anomaly in at least one of the first body part and the second body part.

2. The method of claim 1, wherein the non-ionizing signals are microwaves, that are electromagnetic waves with wavelengths and frequencies in the microwave range.

3. The method of claim 1, wherein the pair of symmetrical body parts is a pair of breasts.

4. The method of claim 1, wherein the anomaly is a presence of malignant tissue.

5. The method of claim 1, wherein comparing the plurality of first parameters with the plurality of second parameters comprises:
generating a first matrix representing the plurality of first parameters;
generating a second matrix representing the plurality of second parameters; and
comparing corresponding entries of the first matrix and the second matrix.

6. The method of claim 1, wherein determining the likelihood of the presence of the anomaly comprises:
in response to a difference between the plurality of first parameters and the plurality of second parameters being less than a threshold, determining that the anomaly is absent the pair of symmetrical body parts; and
in response to the difference between the plurality of first parameters and the plurality of second parameters being greater than the threshold, determining that the anomaly is present in at least one body part of the pair of symmetrical body parts.

7. The method of claim 1, further comprising:
comparing the plurality of first parameters with first historical data representing historical values of the plurality of first parameters; and
comparing the plurality of second parameters with second historical data representing historical values of the plurality of second parameters,
wherein the determining of the likelihood of the presence of the anomaly is further based on the comparison of the plurality of first parameters with the first historical data and the comparison of the plurality of second parameters with the second historical data.

8. A system comprising:
a memory configured to store a set of instructions;
a processor configured to be in communication with the memory, the processor being configured to execute the set of instructions stored in the memory to:
receive a plurality of first parameters corresponding to non-ionizing signals scattered from a first body part among a pair of symmetrical body parts of an individual;
receive a plurality of second parameters corresponding to non-ionizing signals scattered from a second body part among the pair of symmetrical body parts;
compare the plurality of first parameters with the plurality of second parameters; and
based on the comparison, determine a likelihood of a presence of an anomaly in at least one of the first body part and the second body part.

9. The system of claim 8, wherein the non-ionizing signals are microwaves, that are electromagnetic waves with wavelengths and frequencies in the microwave range.

10. The system of claim 8, wherein the pair of symmetrical body parts is a pair of breasts.

11. The system of claim 8, wherein the anomaly is a presence of malignant tissue.

12. The system of claim 8, wherein the processor is configured to:
generate a first matrix representing the plurality of first parameters;
generate a second matrix representing the plurality of second parameters; and
compare corresponding entries of the first matrix and the second matrix to compare the plurality of first parameters with the plurality of second parameters.

13. The system of claim 12, wherein the processor is further configured to store the first matrix and the second matrix in the memory.

14. The system of claim 8, wherein the processor is configured to:
in response to a difference between the plurality of first parameters and the plurality of second parameters being less than a threshold, determine that the anomaly is absent the pair of symmetrical body parts; and
in response to the difference between the plurality of first parameters and the plurality of second parameters being greater than the threshold, determine that the anomaly is present in at least one body part of the pair of symmetrical body parts.

15. The system of claim 8, wherein the processor is configured to:
comparing the plurality of first parameters with first historical data representing historical values of the plurality of first parameters, the first historical data being stored in the memory; and
comparing the plurality of second parameters with second historical data representing historical values of the plurality of second parameters, the second historical data being stored in the memory,
wherein the determination of the likelihood of the presence of the anomaly is further based on the comparison of the plurality of first parameters with the first historical data and the comparison of the plurality of second parameters with the second historical data.

16. An apparatus comprising:
a first device; and
a second device configured to be in communication with the first device, the second device being configured to:
emit a signal on a pair of body parts, the pair of body parts comprises a first body part and a second body part, the first body part and the second body part being symmetrical body parts of an individual;
receive first scattered non-ionizing signals from the first body part;
convert the first scattered non-ionizing signals into a plurality of first parameters;
receive second scattered non-ionizing signals from the first body part; and
convert the second scattered non-ionizing signals into a plurality of second parameters;
wherein the first device is configured to:
receive the plurality of first parameters from the second device;
receive the plurality of second parameters from the second device;
compare the plurality of first parameters with the plurality of second parameters; and
based on the comparison, determine a likelihood of a presence of an anomaly in at least one of the first body part and the second body part.

17. The apparatus of claim 16, wherein the non-ionizing signals are microwaves, that are electromagnetic waves with wavelengths and frequencies in the microwave range.

18. The apparatus of claim 16, wherein the pair of body parts is a pair of breasts.

19. The apparatus of claim 16, wherein the anomaly is a presence of malignant tissue.

20. The apparatus of claim 16, wherein the first device is configured to:
generate a first matrix representing the plurality of first parameters;
generate a second matrix representing the plurality of second parameters; and
compare corresponding entries of the first matrix and the second matrix to compare the plurality of first parameters with the plurality of second parameters.

21. The apparatus of claim 20, wherein the first device is further configured to store the first matrix and the second matrix in a memory.

22. The apparatus of claim 16, wherein the first device is configured to:
in response to a difference between the plurality of first parameters and the plurality of second parameters being less than a threshold, determine that the anomaly is absent the pair of body parts; and
in response to the difference between the plurality of first parameters and the plurality of second parameters being greater than the threshold, determine that the anomaly is present in at least one body part of the pair of body parts.

23. The apparatus of claim 16, wherein the first device is configured to:
compare the plurality of first parameters with first historical data representing historical values of the plurality of first parameters, the first historical data being stored in a memory; and
compare the plurality of second parameters with second historical data representing historical values of the plurality of second parameters, the second historical data being stored in the memory,
wherein the determination of the likelihood of the presence of the anomaly is further based on the comparison of the plurality of first parameters with the first historical data and the comparison of the plurality of second parameters with the second historical data.

24. A method for detecting anomaly in at least one body part among a pair of symmetrical body parts of an individual, the method comprising:
receiving, by a computer device, a plurality of first parameters corresponding to non-ionizing signals scattered from a first body part among the pair of symmetrical body parts;
receiving, by the computer device, a plurality of second parameters corresponding to non-ionizing signals scattered from a second body part among the pair of symmetrical body parts;
comparing the plurality of first parameters with first historical data representing historical values of the plurality of first parameters;
comparing the plurality of second parameters with second historical data representing historical values of the plurality of second parameters; and
based on the comparison, determining, by the computer device, a likelihood of a presence of an anomaly in at least one of the first body part and the second body part.

25. The method of claim 24, wherein the non-ionizing signals are microwaves, that are electromagnetic waves with wavelengths and frequencies in the microwave range.

26. The method of claim 24, wherein the pair of symmetrical body parts is a pair of breasts.

27. The method of claim 24, wherein the anomaly is a presence of malignant tissue.

28. The method of claim 24, wherein:
the first historical data comprises a first set of historical matrices, and comparing the plurality of first parameters with the first historical data comprises:
generating a first matrix representing the plurality of first parameters; and
comparing corresponding entries of the first matrix and the first set of historical matrices; and
the second historical data comprises a second set of historical matrices, and comparing the plurality of second parameters with the second historical data comprises:
generating a second matrix representing the plurality of second parameters; and
comparing corresponding entries of the second matrix and the second set of historical matrices.

29. The method of claim 24, wherein determining the likelihood of the presence of the anomaly comprises:
in response to a difference between the plurality of first parameters and the first historical data being less than a threshold, determining that the anomaly is absent the first body part;
in response to a difference between the plurality of second parameters and the second historical data being less than the threshold, determining that the anomaly is absent the second body part;
in response to a difference between the plurality of first parameters and the first historical data being greater than a threshold, determining that the anomaly is present the first body part; and
in response to a difference between the plurality of second parameters and the second historical data being greater than the threshold, determining that the anomaly is present the second body part.
